(19) United States
(12) Patent Application Publication
(10) Pub. No.: US 2026/0060945 A1
(43) Pub. Date: Mar. 5, 2026

(54) ROOM STABLE HYDROXYUREA ORAL SUSPENSION

(71) Applicant: AKUMS DRUGS & PHARMACEUTICALS LIMITED, Delhi (IN)

(72) Inventors: Sanjeev JAIN, Delhi (IN); Sunil JAISWAL, Delhi (IN); Arun CHAUDHARY, Delhi (IN); Krishna PAL, Delhi (IN)

(73) Assignee: AKUMS DRUGS & PHARMACEUTICALS LIMITED, Delhi (IN)

(21) Appl. No.: 19/104,167

(22) PCT Filed: Feb. 27, 2024
(86) PCT No.: PCT/IN2024/050208

§ 371 Date: Feb. 14, 2025

(30) Foreign Application Priority Data
Sep. 20, 2023 (IN) 202311063613

(51) Int. Cl. A61K 31/17; A61K 9/00; A61K 47/02; A61K 47/14; A61K 47/22; A61K 47/26; A61K 47/44

(52) U.S. Cl. CPC A61K 31/17; A61K 9/0053; A61K 47/02; A61K 47/14; A61K 47/22; A61K 47/26; A61K 47/44

(57) ABSTRACT

The present invention provides a liquid composition comprising effective amount of Hydroxyurea for oral administration. The invention further discloses liquid formulation in form of ready to use oral suspension which remains stable at room temperature at the time of dispensing, storage and even after reopening of the container comprising the suspension. Also provided is a method of preparing the formulation and treating sickle cell anemia in pediatric patients.

### FIELD OF INVENTION

The present invention relates to oral liquid formulation more particularly liquid suspension comprising effective amount of Hydroxyurea which is stable at room temperature and provide process for preparing the same.

### BACKGROUND

Hydroxyurea, also known as Hydroxycarbamide, is an antineoplastic agent which is used in the treatment of chronic myeloid leukemia as well as for sickle-cell disease. The exact mechanism of Hydroxyurea is not exactly known yet but it is believed that it acts by inhibiting DNA synthesis through the inhibition of ribonucleoside diphosphate reductase resulting in cell death in S Phase. It also inhibits repairing of damaged DNA by chemicals or irradiation. Sickle-cell disease in children occurs when children have low level of healthy red blood cells than expected and is a condition in which a child is born with and passed down through parent's genes. Hydroxyurea is a major pharmacological approach for the treatment of sickle cell anemia in children. FDA has approved Hydroxyurea only in tablet and capsule form. However, the challenge with these solid dosage forms were children's acceptance towards this drug delivery. Children who have difficulty in swallowing tablet or capsules or with gastric instability limit the adherence with these solid dosage forms. The poor adherence of solid dosage form results in inadequate disease management. This problem, however, was solved by compounding method. In this method, the oral solution (100 mg/ml) was prepared by emptying the content of 500 mg capsule (20 capsules) and mixing the same in 50 ml sterile water. The resulting solution was stirred for several hours and was mixed in flavoured syrup base and stored in an amber plastic container. This solution was stable till 90 days at room temperature.

However, this liquid solution was compounded unlicensed in pharmacies which limits its availability to the users. Furthermore, poor compounding practice leads to high risk of dosing errors, inadequate efficacy, high risk of adverse reactions, contamination of final formulation and required multiple steps for reconstitution.

To overcome the problem associated with compounding practice, a children friendly, multidose, ready-to-use (no requirement for reconstitution) 100 mg/ml strawberry-flavored oral solution with a stable 2-year shelf life has been developed and marketed as Xromi. Xromi is a patented composition disclosed in patent number EP3644967A1 which covers a stable aqueous hydroxycarbamide solution, a preservative comprising a methyl hydroxybenzoate and/or an ethyl hydroxybenzoate, a sweetener, a viscosity modifying agent, a flavouring agent, a pH adjuster being one of sodium hydroxide, potassium hydroxide, sodium bicarbonate, and sodium carbonate, or a mixture of one or more of these substances, wherein the aqueous hydroxycarbamide solution is controlled to have a pH of between 6.5 and 6.7. The prior art claims stated that that it can be safely stored at ambient temperature for prolonged periods of time without significant degradation however, the formulation was found to be stable at 25° C./60% RH (at room temperature) for only at least two months. Therefore, the marketed formulation Xromi needs to be stored in a refrigerator (2° C.-8° C.) and after its first opening of bottle the unused contents were discarded after 12 weeks. The need of refrigeration causes additional burden to manufacturer, supplier and distributors to ensure that the product is carefully transported, handled and stored in a suitable temperature and to reduce the risk of exposure to temperature outside the labelled storage conditions and also added extra cost to the formulation.

Thus, there still remains a need in art to develop a room temperature stable liquid oral formulation for pediatric patients which overcomes the above mentioned limitation of prior art i.e. compounding medication and need of refrigerating the ready-to-use liquid solution.

### SUMMARY OF THE INVENTION

The present invention provides an oral dosage form such as liquid formulation as well as methods for making such formulation. The liquid formulation of present invention comprises effective amount of Hydroxyurea or pharmaceutically acceptable salts thereof wherein the formulation is stable when kept at room temperature for more than 3 months.

Another aspect of the present invention provides ready-to-use liquid formulation comprising at least 10% Hydroxyurea and one or more pharmaceutically acceptable excipients wherein the selected excipients provide extended shelf-life stability at room temperature when compared to existing marketed formulation.

In yet another aspect, the ready-to-use liquid formulation is selected from solution, suspension, syrup, emulsion or elixir, more particularly, the present invention provides an oral liquid suspension comprising effective amount of Hydroxyurea suspended in a non-aqueous carrier agent.

The inventive liquid suspension of present invention also provides method of preparation thereof wherein the ready-to-use liquid is used to treat sickle cell anemia in pediatric patients who have difficulty swallowing capsule or tablet dosage forms.

### BRIEF DESCRIPTION OF DRAWINGS

FIG. 1 represents linear plot of mean plasma concentrations of Hydroxyurea vs. Time for Test Product (T) and Reference Product (R) (N=28).

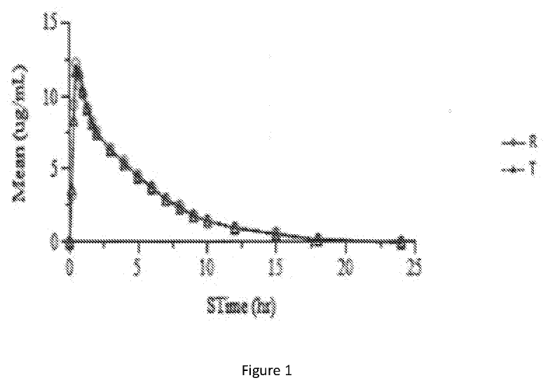

FIG. 2 represents log mean of plasma concentrations of Hydroxyurea Vs. Time for Test Product (T) and Reference Product (R) (N=28).

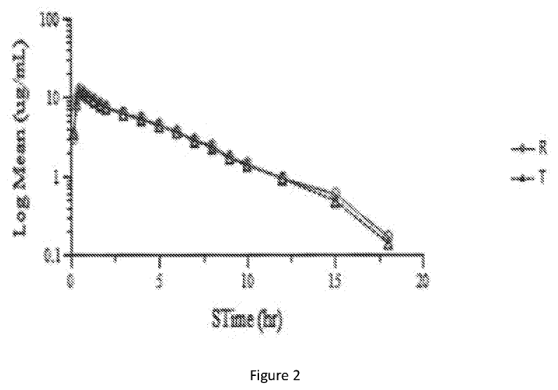

### DETAILED DESCRIPTION OF THE INVENTION

The following paragraphs detail various embodiments of the present invention. For the avoidance of doubt, it is specifically intended that any particular feature(s) described individually in any one of these paragraphs (or part thereof) may be combined with one or more other features described in one or more of the remaining paragraphs (or part thereof).

In other words, it is explicitly intended that the features described below individually in each paragraph (or part thereof) represent important aspects of the invention that may be taken in isolation and combined with other important aspects of the invention described elsewhere within this specification as a whole, and including the examples. The skilled person will appreciate that the invention extends to such combinations of features and that these have not been recited in detail here in the interests of brevity.

The use of the terms “a” and “an” and “the” and similar referents in the context of describing the invention (especially in the context of the following claims) are to be construed to cover both the singular and the plural, unless otherwise indicated herein or clearly contradicted by context.

The term “Hydroxyurea or pharmaceutically acceptable salts thereof” refers to Hydroxyurea in the form of free base or include its salts include but not limited to inorganic or organic salts, hydrates and solvates to a person skilled in the art.

The term pharmaceutically acceptable excipients refer to pharmacologically inactive component such as wetting agent, dispersing agent, co-solvent, sweetener, flavouring agent, preservative, pH modifier, stabilizer, buffering agent, solubilizer and other ingredients of a pharmaceutical product. The excipients which are useful in preparing a pharmaceutical composition are generally safe, non-toxic and are acceptable for veterinary as well as human pharmaceutical use.

The term “anti-oxidant” is used herein to describe any compound or combination of compounds that prevents or slows down Hydroxyurea oxidation.

The term “liquid formulation” refers to drug delivery intended to administered orally generally in form of solutions, suspension, syrup, emulsion or elixir.

The term “room stable” refers to chemical and physical stability of Hydroxyurea for a period of at least one month, particularly for a period of two months, and more particularly for a period of more than three months when stored at room temperature.

The term “ready-to-use” refers to liquid formulation which can be administered to the patients without the need to be compounded or reconstituted at the time of administration or at time prior (e.g., 1 hour, 12 hours, 24 hours, 1 day, 3 days or 7 days) to administration of the formulation.

The present invention is directed to a pharmaceutical composition for oral administration comprising Hydroxyurea or pharmaceutically acceptable salts thereof and a pharmaceutically acceptable excipient in a ready-to-use formulation, i.e., wherein the final composition does not require dilution or compounding or reconstituting prior to administration and is suitable for administration at the point of manufacture, without the need of pharmacist for compounding the formulation.

It is well known that Hydroxyurea is not stable in water for longer period of time when stored at room temperature which is the reason the marketed liquid oral solution needs to be stored in a refrigerator (2° C.-8° C.) and remain stable for 2 or 3 months when kept at room temperature. Unfortunately, none of the presently available liquid dosage form remains stable at room temperature for period not longer than 3 months. Thus, there is a need of liquid dosage form of a Hydroxyurea that maintains the efficacy of the active component, is easily administered to pediatric patients who have difficulty in swallowing solid dosage forms and remains stable at room temperature for longer period of time.

The present invention provides a ready-to-use liquid formulation comprising effective amount of Hydroxyurea or pharmacologically acceptable salts thereof which is suitable for oral administration and which remains stable at room temperature for at least 3 months.

In an embodiment the liquid formulation can be formulated in the form of solutions, suspension, emulsion, syrup or elixirs more preferably in form of ready-to-use liquid suspension.

In an another embodiment the present invention provides a ready to use oral suspension which remain stable at room temperature at the time of dispensing, storage and even after reopening of the container comprising the suspension.

In an another embodiment the present invention provides a ready-to-use oral liquid suspension comprising Hydroxyurea or pharmaceutically acceptable salts thereof wherein Hydroxyurea is present as free base and included in amounts 5 to 15% (w/v) based on total volume of the suspension.

In an embodiment the present invention provides an oral liquid suspension comprising 10% Hydroxyurea suspended in a non-aqueous carrier, more preferably in an edible non-aqueous medium wherein edible non-aqueous medium is a vegetable oil, partially hydrogenated vegetable oil, medium chain triglycerides, free fatty acids, and the likes.

In an embodiment the hydroxyurea is micronized to ensure that hydroxyurea is uniformly dispersed in vehicle and can easily re-dispered on simple shaking.

The micronization also ensure that hydroxyurea remain uniform for sufficient time to ensure uniform withdrawal of dose and thereby ensuring dose accuracy.

In an another embodiment the liquid suspension further comprises at least one excipient selected from wetting agent, a suspending agent, a co-solvent, a sweetener, a flavouring agent, wetting agent, and antioxidants.

The sweetener provides the desired sweetness and palatability to the dosage form and includes but are not limited to sorbitol, dextrose, sodium saccharin, saccharin, glucose, sucralose, trehalose, fructose, xylose, dextrose, maltitol, xylitol, mannitol, aspartame, alitame, neotame or mixtures thereof. Preferred sweeteners are sodium saccharin, saccharin or sucralose.

In a preferred embodiment the sweetening agent is sucralose present in an amount ranging from 0.05-2% w/v more preferably 0.2% w/v.

The flavouring agent enhances and modify the taste and aroma of the dosage form and are thereof included for taste-masking purposes and improving palatability. Flavouring agents for use in particular embodiments include but are not limited to peppermint, lemon oils, vanilla, citrus oil, including lemon, orange, grape, lime and grapefruit or mixtures thereof.

In a preferred embodiment flavoring agents are strawberry and vanilla wherein strawberry flavor is present in an amount ranging from 0.05 to 1% w/v and vanilla flavor is present in an amount ranging from 0.05 to 0.5% w/v.

In an embodiment the wetting agents may select from any suitable water miscible ionic or non-ionic surfactant such as polysorbates, sodium dodecyl sulfate, poloxamer 188, and the likes more preferably Polysorbate.

In a preferred embodiment Polysorbate is present in an amount ranging from 0.05 to 1% w/v more preferably 0.1% w/v.

In an embodiment the formulation of the comprises effective amounts of one or more stabilizers to promote stability of the Hydroxyurea against unacceptable degradation. The stabilizers may comprise one or more antioxidant such as soluble agents such vitamin E, vitamin E acetate, TPGS, propyl gallate, ascorbyl palmitate, butylated hydroxyanisole, butylated hydroxytoluene, monothioglycerol and mixtures thereof.

In anti-oxidant act as stabilizing agent which is used to prevent oxidative degradation in order to obtain a stable suspension having the desired shelf-life and present in an amount effective to stabilize the hydroxyurea contained therein such that hydroxyurea does not degrade to an unacceptable extent and the suspension is deemed to stable for at least 3 months when placed under room temperature.

In a preferred embodiment antioxidant is Vitamin E acetate present in an amount ranging from 0.005 to 0.5% w/v more preferably 0.05% w/v.

In an another embodiment the dispersing/suspending agent is selected from but not limited to calcium phosphate, calcium silicate, colloidal silicon dioxide, hydrophobic colloidal silica, magnesium oxide, magnesium silicate, magnesium trisilicate, and talc most preferably colloidal silicon dioxide.

In a preferred embodiment colloidal silicon dioxide is present in an amount ranging from amount ranging from 1 to 5% w/v more preferably 1.5% w/v.

In an another embodiment the present invention provides ready to stable oral suspension comprising:
i. 5 to 15% w/v Hydroxyurea or pharmaceutically acceptable salts thereof as active ingredient and;
ii. 0.05-2% w/v as sweetening agent and;
iii. 0.05 to 1% w/v flavouring agent and;
iv. 0.05 to 1% w/v as wetting agent and;
v. 0.005 to 0.5% w/v as antioxidant and;
vi. 1 to 5% w/v as dispersing/suspending agent

Wherein the suspension is obtained by dispersing active agent with other excipients in an oily vehicle most preferably a vegetable oil.

In a preferred embodiment the present invention provides ready to use oral suspension comprising:
a. 10% w/v Hydroxyurea or pharmaceutically acceptable salts thereof as active ingredient and;
b. 0.2% w/v Sucralose as sweetening agent and;
c. 0.26% w/v as strawberry dry as flavouring agent and;
d. 0.5% v/v as strawberry as flavouring agent and;
e. 0.32% w/v as Vanilla dry as flavouring agent and;
f. 0.1% w/v as Polysorbate as wetting agent/surfactant and;
g. 0.05% w/v as Vitamin E acetate as antioxidant/stabilizing agent and;
h. 1.5% w/v as Colloidal silicon dioxide as dispersing/suspending agent

The present invention provides a ready-to-use pharmaceutical liquid suspension which have shelf stability longer than 3 months when stored at room temperature, while Hydroxyurea in suspension is generally more chemically and physically stable than commercially available liquid solution.

In another embodiment, the present invention provides a ready-to-use liquid suspension which can easily be administered to pediatric patients for the treatment of sickle cell anemia.

In one embodiment, a room temperature stable formulation which contains no more than total of 6% of impurities formed over the storage period. In one embodiment which contains no more than total of 5% of impurities formed over the storage period more particularly total of 2% impurities formed over the storage period. In one embodiment, the storage period of the formulation of invention can be reasonable period of time in which the formulation has sufficient chemical and physical stability. The storage period can be selected such at least about one month, at least about three months.

In an another embodiment the present invention provides a process of preparing ready to use oral liquid suspension comprising Hydroxyurea. The process of preparing the liquid suspension include mixing Hydroxyurea with one or more excipients wherein the process comprises: dispersing the drug and silicon dioxide with stirring in vegetable oil containing dissolved antioxidant and wetting agent, incorporation with stirring flavor, sweetener, and bulk sweetener and packaging the suspension into suitable container.

In an another embodiment the present invention provides process of preparing room stable suspension comprises following steps:
Preparing the bulk suspension phase by mixing vegetable oil (medium chain triglyceride) with polysorbate, colloidal silicon dioxide, sucralose and vitamin E acetate with continuous stirring for 1 hour to obtain lumpsfree suspension.
Add micronized Hydroxyurea with continuous stirring into bulk suspension obtain from step (a) to form a lumpsfree dispersion and allow the mixing for 01-02 hours.
Add flavouring agent to above solution obtained in step (b) with continuous stirring until lumpsfree dispersion obtained.
Filtering the suspension through a suitable filter followed by filling and sealing in a suitable packaging.

The following examples illustrate the invention and they do not in anyway limit the scope of the invention. A person skilled in the art would easily modify the process for manufacturing the said pharmaceutical composition or could modify the composition with similar materials and finally a person skilled in the art could modify the method of administering the said composition of this invention.

### EXAMPLES

### Example 1: Hydroxyurea Oral Liquid Suspension

TABLE 1
Room stable Hydroxyurea Oral liquid suspension

| Ingredients | Role in formulation | Amount (% w/v) |
|---|---|---|
| Hydroxyurea | Active pharmaceutical | 10.0 |
| | ingredient | |
| Colloidal silicon dioxide | Dispersing agent | 1.5 |
| Sucralose | Artificial sweetener | 0.2 |
| Polysorbate 80 | Wetting agent | 0.1 |
| Strawberry flavor mix | Flavoring agent | 1.1 |
| Vitamin E acetate | Antioxidant | 0.05 |
| Vegetable oil | Vehicle | q.s. to 100.0 |

Disperse hydroxyurea and colloidal silicon dioxide in vegetable oil to obtain a uniform suspension.

### Example 2 Hydroxyurea Oral Liquid Suspension

TABLE 2
Room stable Hydroxyurea Oral liquid suspension

| | | Amount |
|---|---|---|
| Ingredients | Role in formulation | (% w/v) |
| Hydroxyurea | Active pharmaceutical ingredient | 10.0 |
| Colloidal silicon dioxide | Dispersing agent | 1.5 |
| Mannitol | Bulk sweetener | 12.5 |
| Aspartame | Artificial sweetener | 0.32 |
| Polysorbate 80 | Wetting agent | 0.1 |
| Strawberry flavor mix | Flavoring agent | 1.1 |
| Vitamin E acetate | Antioxidant | 0.05 |
| Vegetable oil | Vehicle | q.s. to 100.0 |

### Example 3

TABLE 3
Room stable Hydroxyurea Oral liquid suspension

| | | Amount |
|---|---|---|
| Ingredients | Role in formulation | (% w/v) |
| Hydroxyurea | API | 10% |
| Colloidal silicon dioxide | Suspending agent/dispersing | 1.5% |
| | agent | |
| Sucralose | Sweetening agent | 0.2% |
| Polysorbate 80 | Surfactant | 0.1% |
| Strawberry Dry | Flavour | 0.26% |
| Strawberry | Flavour | 0.526% |
| Vitamin E acetate | Stabilizing agent/antioxidant | 0.05% |
| Vanilla Dry | Flavour | 0.327% |
| Medium chain | Vehicle | q.s. to 100.0 |
| Triglyceride | | |

### Manufacturing Process for Preparing Hydorxyurea Ready to Use Suspension

1. Medium chain triglyceride was mixed with colloidal silicon dioxide, sucralose and vitamin E acetate in a vessel with continuous stirring to obtain bulk suspension phase.
2. Add micronized hydroxyurea with continuous stirring until a lumpsfree dispersion was obtain and allow mixing with bulk suspension obtained in above process step 1.
3. Add suitable flavouring agent with continuous stirring to the suspension obtained in step 2 with continuous stirring until lumpsfree suspension was obtain. Allow soaking and mixing for 2 hours.
4. Filter the whole suspension with nylon cloth and mix again for 15 minutes.
5. Fill the filtered suspension into an amber coloured bottle and seal the bottle.

### Example—4 Stability Test

The sealed bottle of Hydroxyurea suspension prepared from Example—3 was exposed to 25° C.±2° C. and at RH 60%+5% and tested for physical stability such as description, weight and chemical stability such as shelf life and impurities.

TABLE 4
Stability at 25° C. ± 2° C. and at RH 60% ± 5%

| Test | Acceptance Criteria | Initial Test | After 3 months | |
|---|---|---|---|---|
| Description | White to off | White to off | Off white | |
| | white coloured | white coloured | coloured | |
| | suspension filled | suspension filled | suspension filled | |
| | in 60 mL Amber | in 60 mL Amber | in 60 mL Amber | |
| | Pet bottle | Pet bottle | Pet bottle | |
| Weight (gm/ml) | Between 0.90 to 1.20 | 1.049 | 0.9700 | gm/ml |

Each ml contains:

| Hydroxyurea | Shelf life limit | 100.08 mg | 101.17 | mg |
|---|---|---|---|---|
| 100.00 mg | (not less than 90.0 mg & | 100.08% | 101.17% | |
| | not more than 110.0 mg) | | | |
| | (not less than 90.0% & | | | |
| | not more than 110.0% | | | |
| | of label claimed) | | | |

Related substance (by HPLC)

| Single Maximum | Not more than 0.5% | 0.095% | 0.065% |
|---|---|---|---|
| impurity | | | |

| | | | |
|---|---|---|---|
| Total impurities | Not more than 2.0% | 0.141% | 0.065% |

CONCLUSION: The claimed product complies with stability after 03 month of long term stability study (Temp. 25° C. #2° C. & RH 60%±5%)

The sealed bottle of Hydroxyurea suspension prepared from Example—3 was exposed to 30° C.±2° C. and at RH 75%±5% and tested for physical stability such as description, weight and chemical stability such as shelf life and impurities

### Table 5: Stability at Temp. 30° C.≅2° C. & RH 75%±5%

CONCLUSION: The claimed product complies with stability after 03 month of long term stability study (Temp. 30° C. ±2° C. & RH 75%+5%)

### Example—5 Bioequivalence Study

The study evaluated the pharmacokinetic parameters and compared the bioequivalence of Room stable hydroxyurea suspension 100 mg/ml with XROMI (Hydroxycarbamide Oral Solution 100 mg/ml) manufactured by Nova Laboratories Ltd. in healthy, adult, human subjects under fasting conditions.

| Test | Acceptance Criteria | Initial Test | | After 3 months | |
|---|---|---|---|---|---|
| Description | White to off | White to off | | Off | |
| | white coloured | white coloured | | white coloured | |
| | suspension filled | suspension filled | | suspension filled | |
| | in 60 mL Amber | in 60 mL Amber | | in 60 mL Amber | |
| | Pet bottle | Pet bottle | | Pet bottle | |
| Weight/ml | Between 0.90 to 1.20 gm/ml | 1.049 | gm/ml | 0.9699 | gm/ml |
| Each ml contains: | | | | | |
| Hydroxyurea | Shelf life limit | 100.08 | mg | 97.83 | mg |
| 100.00 mg | (not less than 90.0 mg & not | 100.08% | | 97.83% | |
| | more than 110.0 mg) | | | | |
| | (not less than 90.0% & not | | | | |
| | more than 110.0% of label | | | | |
| | claimed) | | | | |

Related substance (by HPLC)

| Single Maximum | Not more than 0.5% | 0.095% | 0.239% |
|---|---|---|---|
| impurity | | | |

| | | | |
|---|---|---|---|
| Total impurities | Not more than 2.0% | 0.141% | 0.317% |

Study: An open label, balanced, randomized, two-treatment, two sequence, two-period, single-dose, two-way crossover, oral bioequivalence study of two formulations of Hydroxyurea Oral Suspension 100 mg/ml of the present invention in comparison with XROMI (Hydroxycarbamide Oral Solution 100 mg/ml) manufactured by Nova Laboratories Ltd. in healthy, adult, human subjects under fasting conditions.

Test Product (T): Hydroxyurea Oral Suspension 100 mg/ml
(present invention

| | |
|---|---|
| Each ml contains: Hydroxyurea IP | 100 mg |

Reference Product (R): XROMI ® (Hydroxycarbamide Oral
Solution 100 mg/ml)

| | |
|---|---|
| Each ml contains: Hydroxycarbamide | 100 mg |

Manufactured by: Nova Laboratories Ltd.

Dose: Oral Suspension 500 mg/5 ml of Test product (T) Hydroxyurea

Oral Suspension IP 100 mg/ml or Oral Solution 500 mg/5 ml of reference product (R) XROMI.

Methodology:

All study related procedures, duration, dates and timings, information on the study treatments

and confidentiality of the subject's data were explained clearly to the subjects by clinical personnel during the informed consent procedure. Subjects who signed the consent form and showed their willingness to participate in the study were enrolled. Subjects who were eligible when assessed against the inclusion and exclusion criteria and who were found to be healthy on physical examination with laboratory investigation values within reference limits were considered for admission to the study. Subjects whose pre-study laboratory values were outside the reference range were also considered for participation provided these values were
considered clinically non-significant by the investigator. Treatments were allocated to subjects as per the Randomization schedule generated by using SAS® software version 9.4 or higher. Blood samples were drawn before dosing (00.00 hour) and up to 24.00 hours after dosing in each period.

Statistical analysis was performed to assess bioequivalence between the pharmacokinetic parameters of test and reference formulations using SAS® software version 9.4. Plasma concentration quantified for Hydroxyurea by using a validated LC-MS/MS.

Number of Subjects:
Subjects Planned: 32 Subjects
Subjects enrolled: 30 Subjects
No. of subjects completed the study: 28 Subjects
No. of subjects withdrawn from the study: 02 Subject (Subject No. 27 in Period I and Subject no. 12 in Period-II)· No. of subjects dropped out: NA
No. of subjects samples bio-analyzed: 30 Subjects
No. of subjects included in pharmacokinetic analysis: 28 Subjects
No. of subjects included in statistical analysis: 28 Subjects
No. of subjects included in safety evaluation: 30 Subjects

Diagnosis and Main Criteria for Inclusion:

Normal, adult, healthy human subjects having age in the range of 18 to 45 years (both inclusive), Body Mass Index between 18.50 to 30.00 Kg/m2 (including both), who were healthy as determined by clinical/medical history, physical examinations, vital sign measurements, clinical laboratory assessments, 12-lead ECG and who met all other inclusion criteria were enrolled in the study.

Method of Administration:

The subjects who administered the IMPs in sitting position in each period, after an overnight fast of at least 10.00 hrs. As per the randomization schedule. Oral suspension dose administration will be done with 240 mL+2 mL of water at ambient temperature by trained personnel, under the supervision of Principal Investigator/authorized trained person in accordance with current version of SOP for, Dosing. The dose was administered in a staggered manner to maintain subsequent blood collection schedule.

Dosing of the Drug Product:

Subjects who receive the test product or reference product were receive the single dose of oral Suspension. The study personnel will turn the syringe upside down thrice to ensure even mixing of the Suspension which will be directly administered into the subject's oral cavity. The subjects were instructed to swallow it with about 50 ml of water from approximately 240±02 mL at ambient temperature. Part of the water (2×05 mL approximately) from the same was used to carefully rinse the dispensed syringe twice. The rinsing and the remaining part of the 240±02 mL water was then given to the subject, thus ensuring complete administration of the dispensed Investigational product.

Duration of Study:

The total duration of the study was 06 days from the day of check-in of the first period till the end of the second period of post study sample.

Criteria for Evaluation:

Assessment of Comparative Bioavailability of the test product with that of reference product was done on the basis of the 90% confidence interval of the geometric mean ratio of Cₘₐₓ and AUC₀₋ₜ between test and reference product obtained after single-dose administration under fasting conditions.

The acceptance criteria for bioequivalence were the entire confidence intervals for the difference of means of Ln-transformed Cₘₐₓ and AUC₀₋ₜ should fall within 80.00-125.00%.

Pharmacokinetic Parameters:

Employing the estimated plasma concentration-time profiles of Hydroxyurea the following pharmacokinetic parameters were calculated using Pheonix® WinNonLin® software version 8.4.

Primary pharmacokinetic parameters: Cₘₐₓ and AUC₀₋ₜ and AUC_{0-inf}

Secondary pharmacokinetic parameters: tₘₐₓ, t_{1/2}, Kₑₗ and AUC % Extra_obs

Statistical Analysis:

Statistical analyses were performed using SAS® software version 9.4. The geometric mean and coefficient of variation was estimated for Cₘₐₓ and AUC₀₋ₜ and AUC_{0-inf}. Arithmetic mean and standard deviation was estimated for AUC % Extrap, tₘₐₓ, t_{1/2}, Kₑₗ. The ANOVA was estimated at alpha 0.05 for log-transformed Cₘₐₓ, AUC0.t and A UCo-inf. The ANOV A model including sequence, treatment and period as fixed effects and subject (sequence) as random effect. 90% confidence intervals for the difference between treatments least-square geometric means were calculated for the log-transformed Cₘₐₓ and AUC₀₋ₜ and AUC_{0-inf}.

The confidence intervals were expressed as percentages relative to the least square geometric means (LSMs) of the reference treatment. The intrasubject variability was calculated using log-transformed pharmacokinetic parameters.

The ratio of least-square geometric means of Test and Reference products was calculated for log-transformed Cₘₐₓ and AUC₀₋ₜ and AUC_{0-inf}.

90% confidence interval (T vs R) for the difference of the least square geometric means of the log transformed values of Cₘₐₓ and AUC₀₋ₜ and AUC_{0-inf} at 5% level of significance should be between 80.00-125.00% for Hydroxyurea.

Safety Assessment:

All subjects who had received at least one dose of investigational product were included in the safety evaluation.

All subjects who had received at least one dose of investigational product were included in the safety evaluation. Safety assessment was based on clinical laboratory evaluation, ECG recordings, clinical examination along with vital signs (body temperature, radial pulse rate, sitting blood pressure and respiratory rate) measurement and post-study clinical laboratory safety evaluation. Laboratory assessments (hematology, biochemistry, serology and urine analysis), and ECG recordings were done at the time of screening. Clinical examination along with vital signs (body temperature, radial pulse rate, sitting blood pressure, respiratory rate) were undertaken at the time of screening, during check-in and before check-out of each period. Vital signs (body temperature, radial pulse rate and sitting blood pressure) questioning for well-being were recorded within 02.00 hours prior to dosing in each period. Post-dose Vital: Well-being assessment, Pulse rate & SBP and DBP were measured, in the sitting position, at the following times: before drug administration and at 02.00, 04.00, 06.00, 12.00, and 24.00 hours after drug administration.

RESULTS:

A) Pharmacokinetic and Statistical Evaluation:

TABLE 6
Descriptive Statistics of Pharmacokinetic Parameters of Test
Product (T) and Reference Product (R) for Hydroxyurea (N = 28)

| | | | | Std | Coeff of | | | |
|---|---|---|---|---|---|---|---|---|
| Treatment | Variable | N | Mean | Dev | variation | Min | Max | Median |
| R | Cₘₐₓ (ng/mL) | 28 | 13.423 | 4.277 | 31.866 | 8.660 | 31.024 | 12.742 |
| | Tₘₐₓ (hr) | 28 | 0.65 | 0.31 | 47.75 | 0.33 | 1.67 | 0.50 |
| | AUC_{0-inf} | 28 | 58.343 | 13.931 | 23.877 | 41.21 | 94.339 | 54.479 |
| | (ng*hr/mL) | | | | | | | |
| | AUC₀₋ₜ | 28 | 54.543 | 13.467 | 24.691 | 37.984 | 89.212 | 50.448 |
| | (ng*hr/mL) | | | | | | | |
| | Kₑₗ | 28 | 0.1965 | 0.0384 | 19.5644 | 0.1154 | 0.2919 | 0.1910 |
| | Thalf (hr) | 28 | 3.66 | 0.76 | 20.64 | 2.37 | 6.01 | 3.63 |
| | AUC_Extrap | 28 | 6.655 | 1.866 | 28.041 | 3.263 | 11.187 | 6.578 |
| | (%) | | | | | | | |
| T | Cₘₐₓ (ng/mL) | 28 | 13.404 | 3.511 | 26.194 | 8.150 | 23.649 | 12.572 |
| | Tₘₐₓ (hr) | 28 | 0.75 | 0.34 | 45.36 | 0.33 | 1.67 | 0.75 |
| | AUC_{0-inf} | 28 | 57.075 | 13.194 | 23.117 | 36.135 | 86.240 | 55.633 |
| | (ng*hr/mL) | | | | | | | |
| | AUC₀₋ₜ | 28 | 53.391 | 12.789 | 23.954 | 31.695 | 82.185 | 51.593 |
| | (ng*hr/mL) | | | | | | | |
| | Kₑₗ | 28 | 0.1980 | 0.0431 | 21.7564 | 0.0999 | 0.2786 | 0.2030 |
| | Thalf (hr) | 28 | 3.69 | 0.97 | 26.28 | 2.49 | 6.94 | 3.41 |
| | AUC_Extrap | 28 | 6.626 | 2.173 | 32.800 | 3.703 | 12.286 | 5.908 |
| | (%) | | | | | | | |

TABLE B
Geometric Least Squares Means, Ratios, 90% Confidence Intervals,
Power and ISCV for Pharmacokinetic Parameters (Cₘₐₓ and AUC₀₋ₜ and
AUC_{0-inf}) of Hydroxyurea (N = 28)

| | Ln- transformed | | | | | | |
|---|---|---|---|---|---|---|---|
| | Geometric Least Square Mean | | | 90% Confidence | | | |
| Pharmacokinetic | Test | Reference | | interval | | | |
| Parameters | product | Product | T/R | (Parametric) | | | ISCV |
| (Units) | (T) | (R) | (%) | Lower | Upper | Power | (%) |
| Cₘₐₓ | 13.0069 | 12.9331 | 100.57 | 93.49% | 108.18% | 99.9 | 16.1 |
| (ng/mL) | | | | | | | |
| AUC₀₋ₜ | 51.9972 | 53.1298 | 97.87 | 93.82% | 102.09% | 100.00 | 9.3 |
| (ng*hr/mL) | | | | | | | |
| AUC_{0-inf} | 55.7020 | 56.9288 | 97.85 | 93.84% | 102.02% | 100.0 | 9.2 |
| (ng*hr/mL) | | | | | | | |

A. Mean Plasma Concentrations Vs. Time Curve

Figure A represents Linear Plot of Mean Plasma Concentrations of Hydroxyurea vs.

Time for Test Product (T) and Reference Product (R) (N=28)

Figure B represents log mean of Plasma concentrations of Hydroxyurea Vs. Time for

Test Product (T) and Reference Product (R) (N=28)

### CONCLUSION

The 90% confidence intervals of the differences of least squares mean for the Ln-transformed pharmacokinetic parameters Cₘₐₓ and AUC₀₋ₜ and AUC_{0-inf} of Hydroxyurea are within the bioequivalence acceptance limits of 80.00-125.00%.

Hence, it is concluded that the Test product (T) Hydroxyurea Oral Suspension IP 100 mg/ml of present invention and Reference product (R) XROMI® (Hydroxycarbamide Oral Solution 100 mg/ml) manufactured by Nova Laboratories Ltd. are bioequivalent with respect to rate and extent of absorption.

1. A stable liquid formulation comprising hydroxyurea or pharmaceutically acceptable salts thereof and at least one pharmaceutically acceptable excipient wherein liquid pharmaceutical formulation is stable when stored at room temperature for longer than 3 months.

2. The stable liquid formulation as claimed in claim 1, wherein the stable liquid formulation is ready to use oral liquid suspension.

3. The stable liquid formulation as claimed in claim 1, comprises Hydroxyurea in an amount 5 to 15% (w/v) based on total volume of the suspension.

4. The stable liquid formulation as claimed in claim 1, comprises Hydroxyurea in micronized form.

5. The stable liquid formulation as claimed in claim 1, comprises at least one excipient selected from wetting agent, a suspending agent, a co-solvent, a sweetener, a flavouring agent, wetting agent, and antioxidants.

6. A ready to use oral suspension as claimed in claim 1, comprising:
a. 5 to 15% w/v Hydroxyurea or pharmaceutically acceptable salts thereof as active ingredient and;
b. 0.05-2% w/v as sweetening agent and;
c. 0.05 to 1% w/v flavouring agent and;
d. 0.05 to 1% w/v as wetting agent and;
e. 0.005 to 0.5% w/v as antioxidant and;
f. 1 to 5% w/v as dispersing/suspending agent;
wherein the suspension is obtained by dispersing active agent with other excipients in an oily vehicle most preferably a vegetable oil.

7. A ready to use oral suspension as claimed in claim 5, comprises:
a. 10% w/v Hydroxyurea or pharmaceutically acceptable salts thereof as active ingredient and;
b. 0.2% w/v Sucralose as sweetening agent and;
c. 0.26% w/v as strawberry dry as flavouring agent and;
d. 0.5% v/v as strawberry as flavouring agent and;
e. 0.32% w/v as Vanilla dry as flavouring agent and;
f. 0.1% w/v as Polysorbate as wetting agent/surfactant and;
g. 0.05% w/v as Vitamin E acetate as antioxidant/stabilizing agent and;
h. 1.5% w/v as Colloidal silicon dioxide as dispersing/suspending agent.

8. A process of preparing a stable liquid formulation of Hydroxyurea wherein the process comprises the steps of:
a) preparing the bulk suspension phase by mixing vegetable oil (medium chain triglyceride) with polysorbate, colloidal silicon dioxide, sucralose and vitamin E acetate with continuous stirring for 1 hour to obtain lumpsfree suspension;
b) adding micronized Hydroxyurea with continuous stirring into bulk suspension obtain from step (a) to form a lumpsfree dispersion and allow the mixing for 01-02 hours;
c) adding flavouring agent to above solution obtained in step (b) with continuous stirring until lumpsfree dispersion obtained; and
d) filtering the suspension through a suitable filter followed by filling and sealing in a suitable packaging.
